(19) United States
(12) Patent Application Publication
(10) Pub. No.: US 2025/0213812 A1
(43) Pub. Date: Jul. 3, 2025

(54) NO delivery device with emergency dosing system

(71) Applicant: INOSYSTEMS, ANTONY (FR)

(72) Inventors: Thierry BOULANGER, ANTONY (FR); Frederic MARCHAL, ANTONY (FR); Akshar PATEL, ANTONY (FR)

(21) Appl. No.: 18/975,632

(22) Filed: Dec. 10, 2024

(30) Foreign Application Priority Data
Jan. 3, 2024 (FR) 2400028

(51) Int. Cl. A61M 16/12; A61M 16/00; A61M 16/10; A61M 16/20

(52) U.S. Cl. CPC A61M 16/12; A61M 16/022; A61M 16/1005; A61M 16/202; A61M 2202/0275

(57) ABSTRACT

The invention relates to an NO delivery apparatus (1) for supplying an NO-containing gas, comprising an NO injection line (111) with a normally closed valve device (113), and a flow rate measurement device (112); an emergency circuit (200) comprising an emergency line (201) connected to the injection line (111), with a normally open emergency solenoid valve (202); and operating means (130). In the event of malfunction of the operating means (130), the flow rate control device (210) supplies the gas at a pre-fixed emergency flow rate of gas that has been determined prior to said malfunction. A multi-way solenoid valve (205) is arranged downstream of the flow rate control device (210) and supplies dosing lines (206, 207) having a calibrated orifice device (208, 209). Installation (1, 2) for supplying gas to a patient and comprising such an apparatus (1).

### CROSS-REFERENCE TO RELATED APPLICATIONS

This patent application claims priority to French Application No. 2400028, filed Jan. 3, 2024 and the entire contents of which are incorporated herein by reference.

### FIELD OF ART

The invention relates to a device for delivering gaseous nitric oxide (NO) to a patient, comprising an NO emergency dosing system and intended to be connected to the patient circuit of a mechanical ventilator, that is to say a medical apparatus for administering gas to a patient, which makes it possible to supply the gas at a pre-fixed flow rate in the event of malfunction, in particular of the operating means.

### BACKGROUND

NO is a gas which, when inhaled, dilates the pulmonary vessels and increases oxygenation by improving gas exchange. It is used to treat various medical conditions such as persistent pulmonary hypertension of the newborn (PPHN), acute respiratory distress syndrome (ARDS) observed mainly in adults, or pulmonary hypertension in heart surgery, as disclosed in particular by EP-A-560928, EP-A-1516639 or U.S. Pat. No. 10,201,564.

Usually, a small quantity of gaseous NO (i.e. a few ppm by volume), diluted in nitrogen (N₂), is injected into a gaseous flow containing oxygen (O₂), which is then inhaled by the patient. The concentration of NO, which corresponds to a dosage, is determined by the physician or similar. Typically, the gas containing O₂ is a mixture of N₂/O₂ or air, such as medical grade air. Generally, the concentration of NO in the gas inhaled by the patient is between 1 and 80 ppm by volume (ppmv), depending on the population treated, i.e. neonates or adults, and therefore the disease to be treated.

The gas inhaled by the patient can be delivered by way of an NO delivery device associated with a mechanical ventilator, as described in U.S. Pat. No. 5,558,083. The NO delivery device is fluidically connected to one or more gas cylinders containing an N₂/NO mixture, of which the concentration of NO can be between typically 200 and 1000 ppmv. Generally, the NO delivery system comprises an NO injection module placed in the inhalation branch of a patient circuit connected fluidically, on the one hand, to the mechanical ventilator and, on the other hand, to a respiratory interface delivering the NO-enriched gas to the patient, for example a breathing mask, a tracheal intubation tube or similar.

The NO delivery system also comprises a flow rate sensor which measures the gaseous flow rate delivered by the mechanical ventilator (i.e. air or N₂/O₂ mixture) in order to determine the quantity of NO to be delivered in order to satisfy the dosage set by the physician.

The NO delivery system can ensure the NO dosage by way of a proportional solenoid valve which delivers a continuous flow of NO-containing gas and which is associated with a flow rate sensor, the two components being arranged in the delivery system, and an injection line connected to the NO injection module, as described in U.S. Pat. No. 5,558,083.

Other systems are available in which the proportional solenoid valve is replaced by a plurality of “all-or-nothing” solenoid valves, delivering the gas intermittently, that is to say in the form of pulses, generally at a high frequency, of which the amplitude and the duration guarantee the correct quantity of gas circulating in the injection line connected to the NO injection module.

In all cases, the known NO delivery systems receive the measurements of the flow rate sensor placed in the inhalation branch of the patient circuit and adjust in real time the quantity of NO to be delivered, according to the desired dosage, by controlling the flow of NO in the injection line.

Since NO is a therapeutically effective agent, that is to say very small concentrations (i.e. a few ppmv) produce a therapeutic effect, its correct dosing is of critical importance, and medical personnel have to constantly adjust the dosage according to the condition of the patient.

When the patient's condition changes, the concentration of NO has to be gradually reduced or increased. For example, in a situation of withdrawal from a neonate whose condition is improving, it is customary to gradually reduce the dosage, for example in steps of 1 ppm, until a zero value is reached, making it possible then to stop the NO delivery system.

Gradual reduction of the NO concentration makes it possible to avoid a “rebound effect”, which may occur in cases of rapid variation of the concentration, for example in cases of abrupt discontinuation of the treatment, the effect being to seriously worsen the condition of the patient.

However, NO delivery apparatuses are sophisticated electrical medical systems which are susceptible to faults or malfunctions that may have a considerable impact on the therapy that is being provided. For example, a major electronic fault or malfunction, in particular of the operating means, may cause a breakdown of the apparatus and therefore a total shut-down of NO delivery, with the negative consequences mentioned above.

Under such circumstances, the NO delivery apparatus has to alert the user, for example by means of an audible alarm signal, that prompt action is required, for example to switch to an emergency pneumatic injection mode, i.e. an “emergency” mode, so as to limit as far as possible the undesirable effects associated with discontinuation of the therapy.

Such switching to an emergency mode is usually done by actuating a control member, such as a rotary knob, that commands for example a continuous delivery of a fixed flow rate of NO, typically of an N₂/NO mixture, for example of the order of 250 ml/min.

However, an emergency dosing mechanism or system is not without risk, in particular for the following reasons:
its activation requires the presence of a person who is authorized to take this action, for example a specialist in neonatology. In a hospital environment, it may take several minutes before this person arrives in the treatment room and thus before the emergency dose is established, and this can entail a momentary discontinuation of the therapy and expose the patient to a rebound effect.
the emergency dose, such as a pre-fixed flow rate of N₂/NO mixture, does not guarantee that the desired dosage is always satisfied. In particular, when the emergency dose is much below the desired dosage, the patient may be exposed to an abrupt change of concentration and may potentially be subject to considerable adverse effects, which is undesirable for obvious reasons of safety and efficacy of the patient's treatment.
the emergency dose is incompatible with some types of ventilators delivering very small volumes, such as high-frequency oscillation (HFO) ventilators, since it may result in an inhaled NO concentration that is too high and that may reach levels that are dangerous to the patient. Therefore, when the patient is being treated with an HFO ventilator, there is no way to administer the NO to the patient, which results in the aforementioned risks associated with the abrupt stoppage of treatment.

It therefore appears that the current emergency dosing mechanisms cannot guarantee a satisfactory level of safety and that it would be desirable for the patient, in the case of an emergency dose being applied due to malfunction of the NO delivery apparatus, to be able to maintain NO therapy without interrupting the therapy and without worrying about the type of ventilator, i.e. HFO ventilator or others, with which the NO delivery apparatus cooperates.

In other words, a problem is to be able to maintain a dosage, that is to say a treatment of the patient by inhaled NO, even in cases of a fault or malfunction of the NO delivery apparatus, in particular a total functional shut-down of the operating means of the NO delivery apparatus, in particular due to a breakdown, a malfunction or a failure of the electrical power supply.

### SUMMARY

A solution according to the invention concerns an NO delivery device or apparatus for supplying an NO-containing gas, typically an NO/nitrogen gas mixture, comprising:
an NO injection line for conveying the NO-containing gas,
a valve device arranged on the injection line to control the circulation of the NO-containing gas in the injection line, said valve device being configured to be normally in a closed position for preventing any circulation of gas in the injection line,
a flow rate measurement device arranged on the injection line in order to perform one or more measurements of the flow rate of the NO-containing gas circulating in the injection line,
an emergency circuit comprising and emergency line which connects fluidically to the injection line upstream and downstream of the valve device, said emergency line comprising an emergency solenoid valve configured to be normally in an open position in order to permit a circulation of gas in the emergency line, and a flow rate control device, and
operating means, i.e. an operating unit, configured to cooperate with the emergency solenoid valve, the flow rate control device, the valve device and the flow rate measurement device.

In the event of a malfunction leading to a halt in cooperation with the operating means, that is to say in the event of a malfunction of the operating means, for example due to a fault in the electrical power supply:
the emergency solenoid valve is configured to pass to an open position in order to permit a circulation of gas in the emergency line of the emergency circuit,
the valve device is configured to pass to a closed position in order to stop all circulation of gas in the injection line, and
the flow rate control device is configured to supply the gas at a pre-fixed emergency flow rate of gas, where said emergency flow rate of gas:
   is determined by the operating means on the basis of at least one gas flow rate measurement supplied by the flow rate measurement device, during a normal functioning of the apparatus prior to said malfunction, and
   is pre-regulated through command of said flow rate control device by the operating means, during said normal functioning of the apparatus.

In addition:
a multi-way solenoid valve is arranged on the emergency line, downstream of the flow rate control device,
said multi-way solenoid valve comprising:
   an inlet path fluidically connected to the emergency line downstream of the flow rate control device,
   a first outlet path fluidically connected to a first dosing line comprising a first calibrated orifice device, and
   a second outlet path fluidically connected to a second dosing line comprising a second calibrated orifice device,
the first dosing line and the second dosing line are connected to the emergency line, downstream of said first and second calibrated orifice devices, and
the operating means are configured to operate the multi-way solenoid valve to direct the gas flow towards the first dosing line or, alternatively, towards the second dosing line.

Depending on the embodiment considered, the apparatus of the invention can comprise one or more of the following features:
the multi-way solenoid valve has 3 paths.
the inlet path of the multi-way solenoid valve is supplied with gas by the emergency line, typically the NO/N₂ mixture, in the event of malfunction of the NO delivery apparatus, in particular of the operating unit, typically in the absence of electrical power supply to said operating unit.
the malfunction causing a cessation of (all) cooperation with the operating means comprises a fault of said operating means or an absence of the electrical power supply to said operating means.
in the event of a malfunction of the NO delivery apparatus, the first outlet path of the multi-way solenoid valve supplies the first dosing line comprising the first calibrated orifice device.
alternatively, in the event of a malfunction of the NO delivery apparatus, the second outlet path of the multi-way solenoid valve supplies the second dosing line comprising the second calibrated orifice device.
the gas inlet path of the multi-way solenoid valve comprises an upstream port receiving the gas, typically an NO/N₂ gas mixture,
the first outlet path of the multi-way solenoid valve comprises a first downstream port supplying the gas, typically an NO/N₂ gas mixture, to the first dosing line.
the second outlet path of the multi-way solenoid valve comprises a second downstream port supplying the gas, typically an NO/N₂ gas mixture, to the second dosing line.
the operating means are configured in order, during the normal functioning of the apparatus, to command the emergency solenoid valve such that the latter is in a closed position preventing any circulation of gas in the emergency line.
the operating means are configured in order, during the normal functioning of the apparatus, to command the valve device to enable a circulation of gas in the injection line and to permit at least one measurement of gas flow rate by the flow rate measurement device.
the operating means are further configured in order, during the normal functioning of the apparatus, to control the flow rate control device in order to pre-regulate the emergency gas flow rate on the basis of at least one gas flow rate measurement supplied by the flow rate measurement device. In other words, the flow rate control device is adjusted before any malfunction, that is to say while the NO supply apparatus is functioning normally.
during the normal functioning of the apparatus, the flow rate measurement device is configured to perform several successive flow rate measurements.
during the normal functioning of the apparatus, the operating means are further configured to determine, for example calculate, the emergency gas flow rate (i.e. the flow rate of gas containing NO, e.g. NO/N₂ mixture) on the basis of one or more flow rate measurements performed by the flow rate measurement device.
the emergency line connects fluidically to the injection line upstream of the valve device, and upstream or downstream of the flow rate measurement device, preferably downstream of the flow rate measurement device.
a flow rate measurement device is arranged on the injection line, upstream or downstream of the valve device, preferably downstream of the valve device,
the emergency line connects fluidically to the injection line via an upstream end, upstream of the valve device, and via a downstream end, downstream of the valve device, so as to bypass said valve device.
the emergency line connects fluidically to an upstream portion of the injection line situated upstream of the valve device, in particular via its upstream end.
the emergency connects fluidically to a downstream portion of the injection line situated downstream of the valve device, in particular via its downstream end.
it comprises storage means for storing at least some of the successive flow rate measurements performed by the flow rate measurement device, that is to say the successive flow rate measurements are stored by storage means.
the storage means are configured to also store one or more lookup tables.
the storage means are configured to also store at least one lookup table giving a relationship between pressure and flow rate of the calibrated orifice device or devices.
the storage means comprise a computer memory, for example a random access memory or other memory.
the NO injection line conveys a gaseous mixture formed of NO and nitrogen, preferably a gaseous mixture NO/N₂ (i.e. nitric oxide/nitrogen) containing between 100 and 2000 ppmv of NO, typically less than 1000 ppmv of NO, the remainder being nitrogen (and, possibly, unavoidable impurities).
the emergency solenoid valve is configured to be normally open, in particular when it is not being operated by the operating means, typically in the event of a malfunction.
the emergency solenoid valve is of the all-or-nothing type.
the operating means comprise at least one microprocessor.
the operating means comprise an electronic board carrying said at least one microprocessor.
the injection line is connected fluidically to a high-pressure line by way of a pressure-regulating device, the high-pressure line and the pressure-regulating device being arranged in the NO delivery apparatus.
the NO delivery device comprises a casing.
the NO emergency dosing system is arranged in the casing, in particular the emergency line and the emergency solenoid valve.
the emergency line connects fluidically to the injection line between the pressure-regulating device and the valve device.
the valve device comprises a solenoid valve, preferably a proportional solenoid valve.
the flow rate control device is configured to form or constitute a system for generating pressure and a proportional flow rate.
the flow rate control device comprises an actuator means cooperating with a pneumatic pressure regulator.
the flow rate control device comprises an actuator means for controlling the output pressure level of the pneumatic pressure regulator.
the flow rate control device comprises an actuator means adjustable by angular displacement.
the actuator means comprises an electric motor, in particular a stepping motor.
the actuator means is powered by the electrical power supply means, i.e. during the normal functioning.
the actuator means comprises an electric motor driving a rotary shaft, integral with the pneumatic pressure regulator.
the pneumatic pressure regulator comprises an inlet port and an outlet port in fluidic communication with the emergency line.
the operating means are configured for operating the actuator means in order to perform a displacement, preferably an angular displacement, of the pneumatic pressure regulator between at least:
   a position of total opening, corresponding to a maximum opening level, that is to say corresponding to a maximum pressure (and maximum flow rate), of the pneumatic pressure regulator. In the position of total opening, the entire gas flow rate supplied by the emergency line enters the pneumatic pressure regulator, that is to say a maximum flow rate.
   a position of total closure, corresponding to a level of total closure, that is to say corresponding to zero pressure (and zero flow rate), of the pneumatic pressure regulator. In the position of total closure, no gas flow rate can pass through the pressure regulator, that is to say a zero flow rate,
   and advantageously at least one intermediate position situated between said positions of maximum opening and of maximum closure, thus corresponding to an output pressure level of the pneumatic pressure regulator lying between the maximum pressure value and the zero pressure value (i.e. 0 bar). In the intermediate position, only some of the gas flow rate conveyed via the emergency line enters the pneumatic pressure regulator, that is to say one or more reduced or limited flow rates below the maximum flow rate.
the operating means are configured for operating the actuator means in order to perform an angular displacement of the pneumatic pressure regulator between several angularly distinct positions, angularly offset from one another, comprising the position of total opening, the position of total closure, and several intermediate positions situated between the positions of total opening and of total closure. Each of said angularly distinct positions corresponds to an output pressure level and to a given gas flow rate, that is to say flow rates lying between the maximum flow rate, the zero flow rate and intermediate flow rates lying between these maximum and zero flow rates.
the operating means are configured to operate, command or control the actuator means during the normal functioning of the device, that is to say before any malfunction, in such a way as to regulate or adjust the pre-fixed emergency flow rate of gas.
the operating means are configured to operate the actuator means in order to perform a displacement, preferably an angular displacement, of a movable element of said actuator means in a given position corresponding to the pre-regulated emergency flow rate.
the movable element comprises a rotary shaft, preferably made of metal or metal alloy.
the mobile element comprises a rotary shaft capable of being driven in rotation by an electric motor.
the operating means are configured to determine the opening of the pneumatic pressure regulator and/or the pre-regulated emergency flow rate on the basis of a stored lookup table.
the operating means are configured to determine an opening of the pneumatic pressure regulator corresponding to the pre-regulated emergency flow rate.
the lookup table is stored by the storage means, such as a computer memory.
it comprises electrical power supply means which are configured to supply electric current to the components that require electrical energy in order to function, in particular the operating means or other components, such as the solenoid valves, the electric motor, etc.
the electrical power supply means comprise means for connection to the mains (110/220V) and/or a battery or similar.
the flow rate control device of the NO emergency dosing system, which forms a proportional system, makes it possible to (pre-) regulate or adjust the pre-fixed emergency flow rate of gas, prior to any malfunction of the apparatus preventing cooperation between the operating means and the emergency solenoid valve, the flow rate control device, the valve device and/or the flow rate measurement device.
the emergency flow rate of gas measured by an NO flow sensor in the NO injection line corresponds to the last flow rate measurement performed by the NO flow rate measurement device prior to the malfunction.
the first calibrated orifice of the first calibrated orifice device has a first passage diameter (D1) and the second calibrated orifice device has a second passage diameter (D2) such that 1.5.D1<D2<4.D1.
the first passage diameter (D1) and the second passage diameter (D2) are such that 1.8.D1<D2<3.D1, preferably with D2 being equal to approximately 2.D1.
the emergency solenoid valve is of the all-or-nothing type, able to assume only an open state, in which it allows the gas flow to pass, and a closed state, in which it interrupts the passage of the gas flow.
the flow rate control device comprises an actuator means cooperating with a pneumatic pressure regulator.
the actuator means comprises a stepping motor, preferably an electric motor.
the pneumatic regulator is configured to be adjustable to several pressure levels between 0 and 2 bar relative, preferably less than 1.5 bar relative.
the actuator means cooperates with the pneumatic regulator to set a desired output pressure downstream of said pneumatic regulator.
the pneumatic regulator comprises an internal spring for adjusting the desired pressure level.
the actuator means comprises a stepping motor configured to adopt several different angular positions, each angular position of the stepping motor corresponding to a given tension of the internal spring of the pneumatic regulator.
the desired output pressure level downstream of the pneumatic regulator is determined by the tension of the internal spring of the pneumatic regulator corresponding to the angular position adopted by the stepping motor.
during normal function, the operating means are configured to operate the multi-way solenoid valve to effect a fluidic communication between the inlet path of the multi-way solenoid valve and either one of the first and second outlet paths of the multi-way solenoid valve so as to have the gas flow pass through the first or second calibrated orifice device.

The invention also relates to an installation for supplying gas to a patient, that is to say a human being, comprising:
at least one NO source containing an NO/N₂ gas mixture,
an NO delivery device according to the invention, supplied with an NO/N₂ gas mixture from said at least one NO source,
an inhalation branch of a patient circuit supplied with the NO/N₂ gas mixture by the NO delivery device, and
a medical ventilator, i.e. a respiratory assistance apparatus, in fluidic communication with the inhalation branch in order to supply said inhalation branch with a respiratory gas containing at least 20% oxygen.

Depending on the embodiment in question, the gas supply installation of the invention can comprise one or more of the following features:
the medical ventilator delivers air or an oxygen/nitrogen mixture, i.e. as respiratory gas containing at least 21% by volume of oxygen.
according to one embodiment, the medical ventilator comprises a motorized blower (i.e. turbine, compressor or similar) delivering the respiratory gas, typically air or an oxygen/nitrogen mixture.
according to another embodiment, the medical ventilator comprises an internal gas circuit comprising one or more proportional valves for conveying the gas and controlling its supply, in particular its flow rate. Such a ventilator is generally supplied with respiratory gas via one or more wall outlets supplied with gas from a network of channels in a hospital establishment or building, typically with air or an oxygen/nitrogen mixture.
the medical ventilator comprises control means, such as one or more electronic control boards.
the control means, such as an electronic control board, operate or control the motorized blower or, depending on the circumstances, the proportional valves of the medical ventilator.
the medical ventilator is of the HFO type or comprises an HFO function, that is to say it is able to produce high-frequency oscillations.
the NO source contains an NO/N₂ gas mixture containing between 100 and 2000 ppmv of NO, the remainder being nitrogen (N₂), stored at a pressure of between 10 and 250 bar absolute, typically at more than 100 bar absolute (before the start of withdrawal).
the NO source contains an NO/N₂ gas mixture containing between 100 and 1000 ppmv of NO, the remainder being nitrogen (N₂), stored at a pressure of between 10 and 250 bar absolute, typically at more than 100 bar absolute (before the start of withdrawal).
the NO source is in the form of one or more pressurized gas cylinders.
the NO source is in the form of one or more gas cylinders having a capacity of between 0.5 and 50 l (water equivalent).
the gas cylinder comprises a cylindrical body made of steel or of aluminium alloy.
the gas cylinder is equipped with a simple valve (without regulator) or one with an integrated regulator.
the gas cylinder is equipped with a regulator valve protected by a protective cap, for example made of metal or polymer.
the patient circuit comprises an inhalation branch and an exhalation branch.
the patient circuit comprises flexible ducts forming the inhalation branch and the exhalation branch, typically hoses made of polymer.
the inhalation branch and the exhalation branch, e.g. flexible ducts, are connected to a joining piece, such as a Y-piece.
the inhalation branch and/or the exhalation branch are connected fluidically to a patient respiratory interface, preferably via the joining piece.
the patient respiratory interface comprises a tracheal intubation tube or a breathing mask.
the inhalation branch and the exhalation branch comprise flexible ducts, for example made of polymer.
the inhalation branch and the exhalation branch are moreover fluidically connected to outlet and inlet orifices, respectively, of the medical ventilator.
the inhalation branch of the patient circuit can comprise a gas humidifier.
the gas humidifier is arranged downstream of the NO injection module in such a way as to be able to humidify the gas before the latter is administered by inhalation to the patient.

According to another aspect, the invention also relates to a method for therapeutic treatment of a person, i.e. a human patient (i.e. adult, child, adolescent or neonate), suffering from pulmonary hypertension and/or hypoxia, which cause pulmonary vasoconstriction or similar, said method comprising administration by inhalation, to the person requiring it, of a gaseous mixture containing from 1 to 80 ppmv of NO and at least 20% by volume of oxygen, preferably at least 21% by volume of oxygen, by means of a gas supply installation, as described above, comprising an NO delivery device equipped with the NO emergency dosing system according to the invention, so as to treat (at least partially) said pulmonary hypertension and/or said hypoxia, which can be caused by one or more pulmonary diseases or disorders such as PPHN (persistent pulmonary hypertension of the newborn) or ARDS (acute respiratory distress syndrome) or can be caused by heart surgery with placement of the patient on extracorporeal blood circulation.

In general, within the context of the invention:
“ppmv” signifies parts per million by volume,
“% vol.” signifies percentage by volume.
“NO” designates nitric oxide.
“NO₂” designates nitrogen dioxide.
“N₂” designates nitrogen.
“O₂” designates oxygen.
the terms “concentration”, “dose” and “content” are considered equivalent.
the terms “operating”, “command” and “control” are considered equivalent and substitutable.
the terms “means of/to/for” are considered to be wholly equivalent to and capable of being substituted by the terms “device of/to/for” or equivalent terms such as “unit”; for example the term “operating means” may be replaced by “operating device” or “operating unit”, the term “measurement means” may be replaced by “measurement device”, etc.
“Normal functioning” is understood to mean the customary functioning of the NO delivery apparatus during a first period of time (of non-zero duration), in the absence of any breakdown, malfunction, fault or the like. The first period of time has a duration of typically one to several minutes, even hours or days, or even more.
“Malfunction” is understood to mean a breakdown, an anomaly, a problem, an incorrect function, a fault or similar, whether electrical, mechanical or of another kind, affecting the normal functioning of the NO delivery apparatus, in particular preventing the functioning of the means of operating the device, during a second period of time (of non-zero duration), for example on account of a breakdown of the operating means and/or a fault in the electrical power supply to the latter. The second period of time has a variable duration, for example from a few seconds to one or more minutes, or tens of minutes, or even more.

### BRIEF DESCRIPTION OF VIEWS OF DRAWINGS

The invention will now be better understood from the following detailed description, which is given by way of non-limiting illustration, with reference to the appended figures, in which:

FIG. 1 is a schematic representation of an embodiment of a gas delivery installation comprising an NO delivery device equipped with an NO emergency dosing system according to the present invention.

FIG. 2 to FIG. 5 are schematic representations of the association between the calibrated orifice and the actuator of the NO emergency dosing system of FIG. 1.

### DETAILED DESCRIPTION

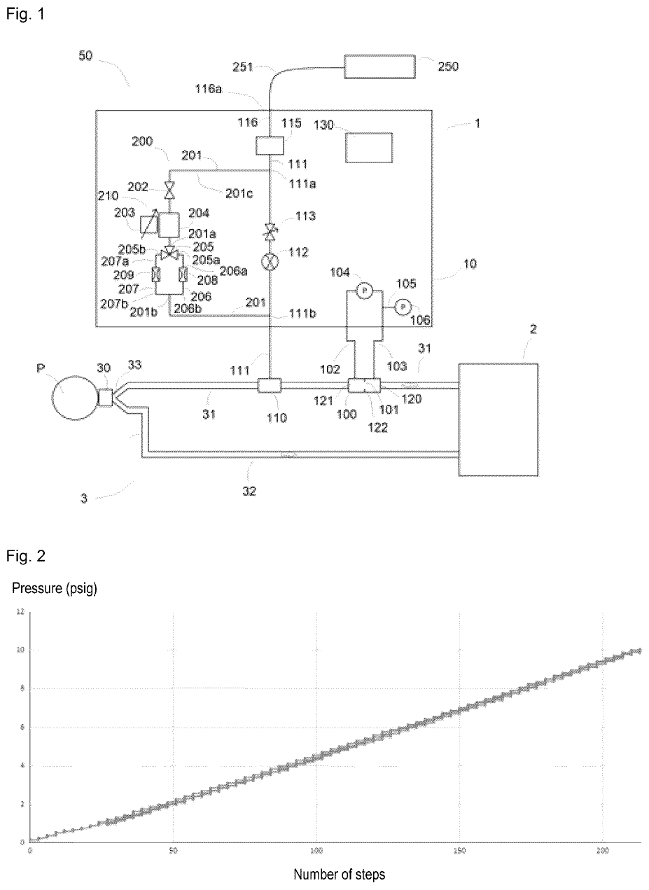

FIG. 1 is a schematic representation of an embodiment of a gas delivery installation 50 according to the present invention, comprising an NO delivery apparatus or device 1, comprising an NO emergency dosing system, associated with a mechanical ventilator 2, that is to say a respiratory apparatus delivering a respiratory gas.

This installation 50 is configured to deliver NO in gaseous form to a patient at a desired concentration corresponding to a dosage set by an anesthetist or the like, typically between 1 and 80 ppmv of NO (i.e. ppm by volume), in particular a flow of NO/N₂ mixture.

The medical ventilator 2 delivers a respiratory gas containing at least about 20% by volume of oxygen, preferably at least about 21% by volume of oxygen, such as air or an O₂/N₂ mixture, into a patient circuit 3, in particular into an inhalation branch 31 of the patient circuit 3, serving to convey and supply the gas to a patient P and to convey the gases exhaled by the patient via an exhalation branch 32 of the patient circuit 3.

The medical ventilator 2 is a conventional respiratory assistance apparatus which, depending on the desired embodiment, can comprise either a motorized blower, also called a turbine or compressor, or one or more proportional valves, in place of the motorized blower, which are supplied with gas, for example with medical air, via a wall outlet supplied by a hospital network that transports the gas within a hospital establishment.

In all cases, when the medical ventilator 2 delivers the respiratory gas into the patient circuit 3, its functioning is controlled by one or more electronic control boards or the like arranged in the medical ventilator 2. It is supplied with electricity by electrical power supply means, such as the mains (110/220V) and/or an internal battery.

For example, the medical ventilator 2 can be the Servo-n Neonatal® from Getinge, which is a ventilator with proportional valves and including an HFO function. Of course, another medical ventilator 2 may also be suitable.

As can be seen from FIG. 1, the inhalation branch 31 and the exhalation branch 32 are fluidically connected to a joining piece 33, such as a Y-piece or similar, in fluidic communication with a respiratory interface 30 for delivering the gas to the patient P or, conversely, for collecting the gases exhaled by the patient P. The respiratory interface 30 can be, for example, a face mask, a tracheal intubation tube or the like.

The inhalation branch 31 and exhalation branch 32 comprise ducts, channels, hoses, passages, tubes or similar, for example flexible hoses made of polymer, which are able and configured to transport the gas flows.

The respiratory gas circulates in the inhalation branch 31 in the direction going from the mechanical ventilator 2 to the patient P, whilst the gases exhaled and enriched in CO₂ circulate in the exhalation branch 32 in the direction of the mechanical ventilator 2, where they are discharged to the atmosphere.

A flow rate sensor 100 and an NO injection module 110 are arranged in the inhalation branch 31. The flow rate sensor 100 is generally arranged between the NO injection module 110 and the mechanical ventilator 2 so as to be able to measure the flow rate of gas coming from the mechanical ventilator 2. The inhalation branch 31 can also comprise a humidifier (not shown) in order to humidify the gas delivered to the patient, which humidifier is preferably arranged downstream of the NO injection module 110, that is to say between the NO injection module 110 and the respiratory interface 30, such as a tracheal intubation tube.

The flow rate sensor 100 serves to measure the flow rate of gas, e.g. air or O₂/N₂ mixture, supplied by the mechanical ventilator 2 and circulating in the inhalation branch 31. The measurements carried out are supplied, directly or indirectly, to the operating means 130 of the NO delivery apparatus 1, which use them to control or adjust the quantity of NO supplied by the NO delivery apparatus 1, that is to say the flow of NO, typically of an NO/N₂ gas mixture, supplied by the NO delivery apparatus to the NO injection module 110, as explained below.

It is possible to use, for example, a mass-flow sensor, a differential pressure sensor or any other suitable sensor.

In the embodiment of FIG. 1, the flow rate sensor 100 is, for example, of the type measuring differential pressure, that is to say the flow rate sensor 100 comprises an internal restriction 101 which creates a drop in pressure, thus generating a pressure differential or gradient when a gas flow passes through this internal restriction 101. The flow rate sensor 100 comprises upstream 120 and downstream 121 chambers which are separated by a wall 122 through which a gas passage extends so as to form the internal restriction 101.

An upstream line 103 and a downstream line 102 for pressure measurement are connected fluidically to the flow rate sensor 100 at connection sites situated upstream and downstream of the internal restriction 101, in particular at the upstream 120 and downstream 121 chambers, in order to perform there the pressure measurements on the circulating flow of gas, before and after a pressure drop, i.e. air or an O₂/N₂ mixture.

The pressure difference created by the internal restriction 101 is determined by a differential pressure sensor 104 connected to the flow rate sensor 100 by way of the upstream line 102 and downstream line 103 which form pressure measurement conduits and supply the differential pressure sensor 104 with the pressure measurements of the circulating flow, before and after a pressure drop.

Preferably, the differential pressure sensor 104 is integrated in the casing 10 of the NO delivery device 1, as is illustrated in FIG. 1.

Moreover, the sensor 104 is connected electrically to an operating unit 130, also designated as controller or operating means, and/or transmits the pressure measurements thereto so that they are processed there by computer, in particular to regulate or adjust the flow rate of NO, typically of NO/N₂ mixture, supplied by the NO delivery apparatus 1 to the NO injection module 110.

The NO injection module 110 injects the flow of NO, i.e. NO/N₂, into the gas flow circulating in the inhalation branch 31 in order to produce the desired mixture therein, that is to say typically an NO/O₂/N₂ mixture containing NO at the desired concentration corresponding to the dosage fixed by a physician or the like, which is typically between 1 and 80 ppmv, generally between 5 and 40 ppmv of NO, the remainder being oxygen (>20% by volume approximately) and nitrogen, even unavoidable impurities (for example argon, etc.) and water vapour, especially when a humidifier is present downstream of the NO injection module 110.

Advantageously, it is possible to provide a branch line 105 which connects to the downstream pressure line 103 in order to carry the information concerning the pressure prevailing in said downstream pressure line 103 to a pressure sensor 106, typically of the relative type. This pressure sensor 106 measures the pressure prevailing in the upstream chamber 120 of the flow rate sensor 100 and can return this value, via an electrical connection, to the operating unit 130 for purposes of compensation, considering that the actual value of the flow rate passing through the flow rate sensor 100 depends principally on the differential pressure measurement 104 but is also affected by the relative pressure 106 prevailing upstream of the flow rate sensor 100.

The operating unit 130 comprises a system for processing data, in particular measurements coming from the sensors 100, 104, 106, typically comprising one or more microprocessors arranged on one or more electronic boards and using one or more algorithms, i.e. one or more computer programs. Of course, the operating unit 130 can also be configured to operate other electromechanical elements integrated in the casing 10 of the NO delivery device 1.

More precisely, the operating unit 130 is configured to process and/or exploit the measurements, that is to say the pressure measurement signals or the pressure values, transmitted by the differential pressure sensor 104 cooperating with the flow rate sensor 100, and/or by the pressure sensor 106. Advantageously, the control unit 130 has a pre-recorded, i.e. stored, lookup table which permits determination of the flow rate of gas circulating in the inhalation branch 31, i.e. passing through the flow rate sensor 100, that is to say makes it possible to transform a pressure value transmitted by the pressure sensor 104, here a differential pressure sensor 104, into a value of the flow rate passing through the flow rate sensor 100, optionally compensated by the value returned by the pressure sensor 106.

In general, the determination of the flow rate of the gas (e.g. air) passing through the flow rate sensor 100 then makes it possible to calculate the quantity of NO (i.e. the flow rate of NO/N₂) that has to be injected by the NO injection module 110 into the gas flow circulating in the inhalation branch 31, so as to be able to deliver the NO to the patient at the desired concentration corresponding to the dosage fixed by an anesthetist or similar, typically between 1 and 80 ppmv of NO (i.e. ppm by volume).

In other words, using the pressure measurement returned by the differential pressure sensor 104 and the stored lookup table, the operating unit 130 is able to determine the flow rate of gas (e.g. air or N₂/O₂ with content of O₂>21% by volume) issuing from the mechanical ventilator 2 and the quantity of NO that has to be added, via the NO injection module 110, in order to obtain the desired concentration of NO.

As already mentioned, the final gaseous mixture obtained at the NO injection module 110 then principally comprises nitrogen (N₂), oxygen (O₂) in a content of at least 20 to 21% by volume, and NO in a content of typically between 1 and 80 ppmv, and even unavoidable impurities and/or water vapour, especially when a gas humidifier is present.

More precisely, depending on the gaseous flow rate (i.e. air or N₂/O₂) circulating in the inhalation branch 31 and determined with the aid of the flow rate sensor 100, the operating unit 130 determines the quantity of NO, typically an NO/N₂ mixture, that has to be added to the gas with an O₂ content>20% by volume (e.g. air or N₂/O₂) circulating in the inhalation branch 31 in order to obtain the desired final concentration of NO.

The NO delivery apparatus 1 is supplied with gaseous NO, typically as a gaseous mixture NO/N₂, coming from an NO source 250 connected fluidically to the NO delivery device 1, in particular to a high-pressure line 116 of said NO delivery device 1, via a supply line 251, such as a flexible conduit or similar. Typically, the NO source 250 is in the form of one or more pressurized gas cylinders holding a mixture of NO/N₂ containing a concentration of NO of generally between 100 and 30000 ppmv.

The NO/N₂ mixture is supplied to the injection module 110 by the NO delivery apparatus 1 via an injection line 111, such as a flexible gas conduit, which is fluidically connected to the high-pressure line 116 of the NO delivery apparatus 1, which high-pressure line has a high-pressure inlet 116a connected fluidically to the NO source in order to be supplied with NO/N₂ under pressure, e.g. 10 bar abs.

The high-pressure line 116, for example a gas passage or conduit, comprises a pressure regulator 115 which reduces the pressure of the NO/N₂ mixture to a stable value, for example about 2 bar abs or any other suitable pressure. The outlet port of the pressure regulator 115 thus provides a stable pressure in the upstream portion of the injection line 111.

A valve device 113, such as a solenoid valve, advantageously a proportional solenoid valve, for example the solenoid valve of the miniature VSO series from Parker, is arranged in the NO delivery apparatus 1 in order to control the flow rate of gaseous NO within the injection line 111.

The flow rate of gas circulating in the injection line 111 is measured by a flow rate measurement device or NO flow rate sensor 112 arranged on the injection line 111, preferably placed downstream of the valve device 113, as can be seen in FIG. 1.

The pressure regulator 115, the valve device or solenoid valve 113, the NO flow rate sensor 112 and an upstream portion of the injection line 111 are thus arranged in the casing 10 of the NO delivery apparatus 1.

The valve device 113 is configured to be normally in a closed position (i.e. a closed state) in order to prevent all circulation of gas in the injection line 111. To pass to the open position, the valve device 113 has to be controlled by the operating means 130, as is the case during normal functioning of the NO delivery apparatus 1.

Moreover, an NO emergency dosing system is provided, that is to say an emergency circuit 200, which is arranged in the casing 10 of the NO delivery apparatus 1 and which is configured to function in the event of a malfunction of the NO delivery apparatus 1, as is explained below.

The emergency circuit 200 comprises (at least) an emergency line 201, also called a bypass line, such as a gas passage or conduit or the like.

In the embodiment proposed in FIG. 1, the emergency line 201 of the emergency circuit 200 connects fluidically to the injection line 111 at a first connection site 111a, i.e. upstream, situated between the pressure regulator 115 and the valve device 113, and at a second connection site 111b, i.e. downstream, situated downstream of the valve device 113 and, preferably, downstream of the NO flow rate sensor 112.

In other words, the valve device 113 and preferably the NO flow rate sensor 112 are situated between the first and second connection sites 111a, 111b of the emergency line 201, that is to say the emergency line 201 bypasses the valve device 113 and preferably the NO flow rate sensor 112, which are arranged on the injection line 111.

Alternatively, according to another embodiment, the second connection site 111b can be situated between the valve device 113 and the NO flow rate sensor 112.

In all cases, the gas circulates in the emergency line 201 in the direction from the first connection site 111a to the second connection site 111b.

The emergency circuit 200 also comprises an emergency solenoid valve 202 and a flow rate control device 210 which are arranged on the emergency line 201 and which serve to control the flow rate of gas within the emergency circuit 200, typically within the emergency line 201.

A 3-way solenoid valve 205, that is to say of the 3:2 type, is arranged on the emergency line 201, downstream of the flow rate control device 210. It is controlled by the operating means 130.

The 3-way solenoid valve 205 comprises an upstream port 201a, a first downstream port 205a and a second downstream port 205b, in fluidic communication. The fluidic communication between the upstream port 201a and the first downstream port 205a or, alternatively, the second downstream port 205b is selected by the operating means 130, as described below.

The 3-way solenoid valve 205 is of the bistable type, that is to say in the event of no electrical control by the operating means 130, for example in the event of malfunction, typically in the event of a loss of electrical power, the fluidic communication existing between the upstream port 201a and the first downstream port 205a or the second downstream port 205b is maintained, that is to say it remains in the state in which it was prior to malfunction.

By way of example, it is possible to use the solenoid valve 205 designated HDI and available from the Lee Company®.

The first downstream port 205a of the solenoid valve 205 is in fluidic communication with a first dosing line 206 in which a first calibrated orifice device 208 is arranged, whereas the second downstream port 205b of the solenoid valve 205 is in fluidic communication with a second dosing line 207 in which a second calibrated orifice device 209 is arranged.

The first and second calibrated orifice devices 208, 209 have different characteristics in terms of the passage cross section of their respective calibrated orifices, e.g. different diameters. Thus, the first calibrated orifice of the first calibrated orifice device 208 can have a first passage diameter D1 and the second calibrated orifice device 209 has a second passage diameter D2, such that D1<D2, preferably 1.5.D1<D2<4.D1.

For example, the first calibrated orifice of the first calibrated orifice device 208 can have a first passage diameter D1 of the order of 25 μm and the second calibrated orifice of the second calibrated orifice device 209 has a second passage diameter D2 of the order of 50 μm, that is to say that D2 is preferably equal to about 2.D1.

By way of example, it is possible to use calibrated orifices available from the company O'Keefe Control® under the designations BLP-1-SS and BLP-2-SS. Of course, it is possible to use other calibrated orifices of different diameters and/or shapes.

Downstream of the first and second calibrated orifice devices 208, 209, the first and second dosing lines 206, 207 meet at a connection site 201b, called a node, and are furthermore connected, at this same site 201b, to the downstream part of the emergency line 201, which emergency line 201 is fluidically connected (downstream of the site 201b) to the injection line 111 at the level of the second connection site 111b. In other words, the first dosing line 206 and the second dosing line 207 are connected at the node 201b so as to form the downstream part of the emergency line 201.

The emergency solenoid valve 202 is configured to be normally in an open position (i.e. open state) to permit circulation of gas in the emergency line 201, that is to say it allows the gas flow to pass when it is not or no longer controlled by the operating unit 130. During normal functioning of the NO delivery device 1, the emergency solenoid valve 202 is thus controlled by the operating means 130 to be in a closed position (i.e. a closed state) in order to prevent the flow of NO/NO₂ from following the emergency line 201.

The emergency solenoid valve 202 is preferably a solenoid valve of the “all-or-nothing” type having two possible states, namely an open state, allowing the gas flow to pass, and a closed state, not allowing the gas flow to pass. It is controlled by the operating unit 130. It is possible to use, for example, a solenoid valve of the Picosol series from IMI Norgren®, or of the HDI series from the Lee Company®.

As has already been mentioned, the emergency solenoid valve 202 is normally open, that is to say, in the absence of an electrical control from the operating unit 130, the emergency solenoid valve 202 is in the open state, i.e. open position, which then allows the gas coming from the NO source to follow the emergency line 201 from the first connection site 111a in the direction of the second connection site 111b.

On the other hand, the operating unit 130 controls the closure of the emergency solenoid valve 202, that is to say the changing of the latter from the open state to the closed state, i.e. the closed position, in order to prevent any circulation of gas in the emergency line 201, especially when such a flow is not desired, typically during normal functioning.

In other words, the operating means 130, i.e. the operating unit, are configured to cooperate with the emergency solenoid valve 202, the flow rate control device 210, the valve device 113 and the flow rate measurement device 112, during normal functioning of the NO delivery apparatus 1, in order to direct the gas flow to the injection line 111 and prevent it from circulating in the emergency line 201, and vice versa in the event of a malfunction, as is explained below.

In the embodiment of FIG. 1, the flow rate control device 210 comprises an actuator means 203, preferably adjustable by angular displacement, typically a stepping motor, cooperating with a pneumatic pressure regulator 204, and thus forms a variable pressure system making it possible to control the flow rate and the pressure of the gas flow.

When the emergency solenoid valve 202 is open, that is to say not controlled by the operating unit 130, typically in the event of a supply fault or malfunction of the operating unit 130, the pressure prevailing in the upstream portion 201c of the emergency line 201 (i.e. between the first connection site 111a and the pneumatic regulator 204) is equal to the expansion pressure of the pressure regulator 115, for example here equal to 2 bar rel. (14 psig). This same pressure is also exerted at the inlet of the pneumatic regulator 204, which is arranged on the emergency line 201.

For its part, the pneumatic regulator 204 can be adjusted to several different pressure levels, typically up to 2 bar relative, for example between about 0 and 1.4 bar relative (i.e. 0-20 psi), depending on the tension of its internal spring. For example, it is possible to use a pneumatic regulator available from Beswick Engineering® under the designation PRDB.

The actuator means 203, namely in this case a stepping motor, is mechanically coupled to the pneumatic regulator 204 in such a way that a given angular position of the stepping motor influences the tension of the internal spring of the pneumatic regulator 204 and thus fixes an outlet pressure downstream of said pneumatic regulator 204.

In other words, depending on its angular position determined by the operating means 130, the stepping motor can control the pneumatic regulator 204, in particular by acting on the tension of its internal spring, to pass from a closed position, i.e. delivering a zero pressure at its outlet, to an open position, delivering a maximum pressure at its outlet, typically less than 2 bar relative, for example of the order of 1.4 bar relative (i.e. approximately 20 psig).

Of course, depending on the angular position of the stepper motor, that is to say as a function of its “number of steps”, the pneumatic regulator 204 can adopt one or more intermediate positions, thus delivering a pressure comprised between a minimum, for example 0 bar relative, and a maximum typically less than 2 bar relative, for example 1.4 bar relative.

Consequently, the resolution in terms of adjustable pressures downstream of the pneumatic regulator 204 depends on the fineness and the number of steps defining a given position of the stepping motor, i.e. of the actuator means 203, at its next position.

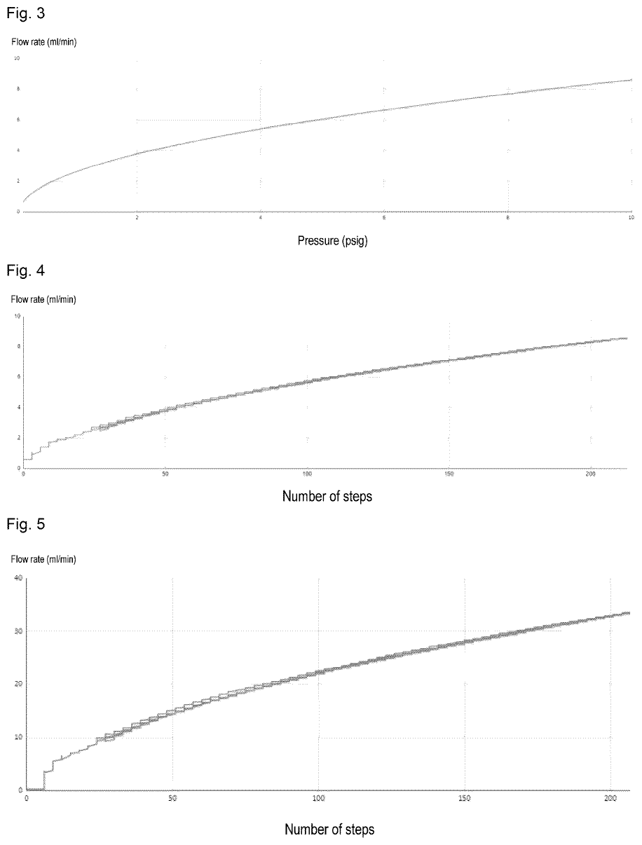

Thus, FIG. 2 shows an evolution of the output pressure of the pneumatic regulator 204 as a function of a number of steps (i.e. position of the stepping motor), determined by the operating means 130, which makes it possible to observe that the pressure increases linearly as a function of the number of steps. More precisely, it can be seen that the maximum output pressure of the pneumatic regulator 204 is limited to 12 psig (830 mb rel.) but that it could be higher, typically up to 20 psig (1.4 bar rel.), by increasing the number of steps.

More generally, the output pressure of the pneumatic regulator 204 is then at the level of the upstream port 201a of the solenoid valve 205, which is arranged on the emergency line 201 downstream of the pneumatic regulator 204.

During normal functioning, the multi-way solenoid valve 205, here a three-way solenoid valve, is also controlled by the operating means 130 to effect a fluidic communication between its inlet path, which is fluidically connected to the emergency line 201 downstream of the flow rate control device 210, and either one of its first outlet path, which is fluidically connected to the first dosing line 206, and its second outlet path, which is fluidically connected to the second dosing line 207, that is to say between its upstream port 201a and one of its downstream ports 205a, 205b.

The output pressure of the pneumatic regulator 204 then propagates in the upstream portion 206a of the first dosing line 206 situated upstream of the first calibrated orifice 208 or, as the case may be, in the upstream portion 207a of the second dosing line 207 situated upstream of the second calibrated orifice 209.

In other words, during the normal functioning of the apparatus 1, the operating means 130 control the 3-way solenoid valve 205 to effect a fluidic communication between the inlet path of said solenoid valve 205 and one or other of the first and second outlet paths of the 3-way solenoid valve 205, so as to cause the gas flow to circulate in one or other of the dosing lines 206, 207, hence through the first or second calibrated orifice device 208, 209 which comprise different flow cross sections or diameters of their calibrated orifices D1, D2.

However, in general, for any calibrated orifice, there is a relationship between the pressure upstream of the calibrated orifice and the flow rate passing through it, since the flow rate depends on the dimensions of the orifice in question. In fact, the flow rate passing through a calibrated orifice is related to the pressure differential existing between the pressure upstream and the pressure downstream of this calibrated orifice.

Thus, FIG. 3 shows the relationship linking the pressure (in psig) upstream of the first orifice of the first calibrated orifice device 208 and the flow rate (in ml/min) passing through it, i.e. circulating in the downstream portion 206b of the first dosing line 206.

It can be seen that the flow rate increases as the pressure upstream increases. This increase does not follow a linear law but rather one of the “square root” type, as widely documented in the literature.

Consequently, here, as a function of the position of the solenoid valve 205, the flow rate passing through the first calibrated orifice of the first calibrated orifice device 208 depends on the difference in the pressures prevailing respectively in the upstream portion 206a and downstream portion 206b of the first dosing line 206, and, conversely, the flow rate passing through the second calibrated orifice of the second calibrated orifice device 209 depends on the difference in the pressures prevailing respectively in the upstream portion 207a and downstream portion 207b of the second dosing line 207.

Preferably, the pressure prevailing downstream of the first and second calibrated orifices, that is to say downstream of the two calibrated orifice devices 208, 209 (i.e. in the downstream portions 206b, 207b), is also considered to be negligible. However, additional measuring means, such as an additional pressure measurement device, may be arranged to perform a pressure measurement downstream of the first and second calibrated orifices, for example in the region of the node 201b of the emergency line 201, and used for pressure compensation purposes in order to increase the accuracy of the flow rate control device 210, as is set out in detail below.

Furthermore, the expression of the flow rate also corresponding to a position of the stepping motor 203, expressed in the form of steps, as shown in FIG. 4, based on the linear relationship between the position of the motor 203 and the output pressure of the pneumatic regulator 204, as shown in FIG. 2.

Thus, FIG. 4 shows that a number of steps equal to 0 corresponds to a closed position of the pneumatic regulator 204 and each step corresponds to a change of position of the stepping motor 203 opening the pneumatic regulator 204 a little more to allow the gas flow to pass. For example, for 50 steps taken, a flow rate of about 4 ml/min is obtained, while for 100 steps taken, the flow rate is about 5.5 ml/min.

Thus, by informing a lookup table linking a number of steps (i.e. a position of the stepping motor 203) and a resulting flow rate, the operating means 130 can “(pre-) regulate” the emergency dosing system or circuit 200, during the normal functioning of the apparatus 1, as explained below, so that it is operational in the event of a malfunction of the operating unit 130, typically in the event of a supply failure of the control unit 130.

In other words, during the normal functioning of the apparatus 1, the operating means 130 control the pneumatic regulator 204 to adjust or fix the position of the stepping motor 203 at a determined number of steps corresponding to a desired flow rate of gas.

Similarly, during the normal functioning of the apparatus 1, when the operating means 130 control the 3-way solenoid valve 205 to effect a fluidic communication between its upstream port 201a and, for example, its second downstream port 205b, it is possible to establish, as previously, a lookup table linking a number of steps (i.e. a position of the stepping motor 203) and a resulting gas flow rate then circulating in the downstream portion 207b of the second dosing line 207, as is illustrated in FIG. 5.

Given that the diameter of the second calibrated orifice 209 is greater than that of the first calibrated orifice 208, the flow rate resulting at a similar “step” (i.e. similar position) is greater. For example, for 50 steps taken, a flow rate of about 15 ml/min is obtained (4 ml/min for the first calibrated orifice 208).

In other words, depending on the configuration of the 3-way solenoid valve 205, the operating unit 130 can have a lookup table linking a given control level (i.e. of steps) to a flow rate of gas passing through the first or, alternatively, the second calibrated orifice 208, 209 in the direction of the injection line 111 and entering the latter at the second connection site 111b.

All these (pre-) adjustments are carried out during the correct functioning of the apparatus 1, that is to say during its normal functioning before any malfunction of the operating unit 130, in particular when it is no longer supplied with electric current and therefore no longer functions.

This is then used to ensure the delivery of an emergency NO flow (i.e. NO/N₂ flow), even in the event of malfunction of the operating unit 130, that is to say when it is no longer supplied with electric current and therefore no longer functions, since all the adjustments have already been made, before the malfunction.

Thus, during normal functioning of the NO delivery apparatus 1, that is to say when the operating unit 130 is operational and normally supplied with electric current, the emergency solenoid valve 202 is controlled by the operating unit 130 to be closed, which prevents any circulation of gas in the emergency circuit 200 of FIG. 1, whereas in the event of a malfunction of the apparatus 1 rendering the operating unit 130 non-operational, such as an electrical fault, the emergency solenoid valve 202 can no longer be controlled by the operating unit 130 and therefore opens to allow the gas to pass into the emergency circuit 200, while the solenoid valve 113 closes, as has already been explained. The gas flow circulating in the emergency circuit 200 is then subjected to the (pre-) adjustments made before the malfunction, i.e. position of the stepping motor, sending of the flow to the first or second calibrated orifice device 208, 209, etc.

In general, using a stepping motor as actuator means 203 is particularly recommended since, in contrast to the solenoid valves 202, 113 which will adopt a rest position if the power is cut, namely an open position for the all-or-nothing solenoid valve 202 and a closed position for the proportional solenoid valve 113, the position of the stepping motor does not change, i.e. remains permanent and fixed according to the last command imposed, and this independently of any electrical supply.

In other words, the tension of the internal spring of the pneumatic regulator 204 has a fixed value equal to the last control value coming from the operating means 130 and received by the stepping motor, that is to say a given position corresponding to a given number of steps, during the normal functioning of the apparatus 1.

If the pneumatic regulator 204 is supplied with gas, that is to say when the emergency solenoid valve 202 opens due to an absence of control by the operating means 130, the tension of the internal spring of the pneumatic regulator 204 generates a fixed pressure downstream of the pneumatic regulator 204, which is then at the upstream port 201a of the solenoid valve 205.

Of course, the present invention is not limited to an actuator in the form of a stepping motor. In fact, it is possible to use any other actuator that keeps its position if the electrical power supply fails and that can be coupled to a mechanical mechanism so as to define or constitute a variable-pressure system, for example a linear motor or similar.

In general, during its normal functioning, the NO delivery device 1 is furthermore powered electrically by an electrical power supply, for example the mains (110/220V) or an internal battery, in order to permit the correct functioning of the components thereof that require electric current in order to function, in particular the actuator 203, such a stepping electric motor, the operating unit 130, the solenoid valves 202, 113, 205 or others.

In addition, the NO delivery device 1 also comprises storage means, such as a computer memory, for storing data, information or the like, for example one or more lookup tables, as explained above, the gas flow rate measurements performed by the flow rate measurement device 112, or others.

In general, in the event of the NO delivery device 1 suffering a major failure, such as a failure of its electrical power supply, caused for example by its electrical power supply cable being ruptured by vibrations during patient transport for example, one must be able to continue providing treatment to the patient with inhaled NO, this despite the malfunction generating a shut-down of the functioning of the operating means 130, typically on account of an electrical power supply fault.

To this end, the device 1 of the invention is configured such that, in the event of such a fault, the emergency solenoid valve 202 passes to an open position in order to allow gas to circulate in the emergency line 201, while at the same time the valve device 113 passes to a closed position in order to stop any circulation of gas in the injection line 111, which makes it possible to supply the gas, via the emergency line 201 and the flow rate control device 210, at a pre-fixed emergency flow rate of gas.

In fact, during the normal functioning of the device 1 prior to the malfunction, said emergency flow rate of gas is determined by the operating means 130 on the basis of one or more gas flow rate measurements supplied by the flow rate measurement device 112 to the operating means 130. The operating means 130 can then pre-regulate the flow rate control device 210 and the solenoid valve 205 so that they can deliver the gas at the pre-fixed emergency gas flow rate.

In other words, the operating means 130 determine the emergency flow rate of gas, which has to be administered in the event of a breakdown or other malfunction, on the basis of the gas flow rate measurements supplied by the flow rate measurement device 112 during the normal functioning of the device 1 and act on the flow rate control device 210 and the 3-way solenoid valve 205 in order there to regulate this pre-fixed emergency flow rate of gas, for example by acting on the pneumatic regulator 204, as is explained above.

More generally, the functioning of the gas delivery installation 50 comprising the NO delivery device 1 of the invention is as follows.

As is illustrated in FIG. 1, the NO delivery device 1 cooperates with a mechanical ventilator 2 in order to provide therapeutic aid to the patient P. As has already been explained, the flow rate of gas (i.e. air or N₂/O₂) issuing from the mechanical ventilator 2 and circulating in the inhalation branch 31 of the patient circuit 3 is measured permanently by the flow rate sensor 100 and the operating unit 130. The flow rate measurement(s) performed by the flow rate sensor 100 allow(s) the operating unit 130 to determine, in real time, the NO flow rate that has to circulate in the injection line 111 as far as the NO injection module 110 in order to inject the quantity of NO into the air flow coming from the ventilator 2 so that the desired final NO concentration can be obtained, typically between 5 and 80 ppmv, in the final NO/O₂/N₂ gas mixture delivered to patient P.

During normal functioning, that is to say in the absence of a breakdown or malfunction, so as not to introduce an additional flow coming from the emergency line 201 into the injection line 111, the operating unit 130 controls the solenoid valve 202, which is preferably of the all-or-nothing type, in the closed position and, in parallel, will operate the actuator 203, such as a stepping motor, in order to pre-regulate the pneumatic regulator 204 by defining a level of tension of its internal spring, preferably as a function of the position adopted by the stepping motor 203, as has been explained above.

This is done by the operating unit 130 on the basis of one or more flow rate measurements coming from the flow rate measurement device 112.

More precisely, the operating unit 130 first of all establishes a mean flow rate of NO (i.e. of the NO/N₂ mixture) that has circulated in the injection line 111 for a given time, for example for 1 minute, or for a longer period of time (but the flow rate must then be converted to l/min or else ml/min)), during normal functioning of the device 1.

The operating unit 130 therefore estimates a fixed mean flow rate of NO (in l/min or ml/min) making it possible to approach the desired concentration of NO.

Thus, in Table 1, the fixed mean flow rate of NO (in ml/min) is given for different selected concentrations of NO (in ppmv), i.e. dosages, resulting from different minute ventilations (l/min) measured by the flow rate sensor 100, which the operating means 130 use to determine the flow rate of NO to be delivered in real time.

TABLE 1

| NO content (dosage) | Minute ventilation (l/min) | | | |
|---|---|---|---|---|
| (in ppmv) | 2 | 4 | 6 | 15 |
| 5 | 0.1 | 0.2 | 0.3 | 0.8 |
| 10 | 0.5 | 1 | 1.5 | 3.8 |
| 20 | 1 | 2 | 3 | 7.5 |
| 40 | 2 | 4 | 6 | 15 |
| 60 | 4 | 8 | 12 | 30.1 |
| 80 | 6 | 12 | 18.1 | 45.1 |

It is found that for a mean minute ventilation of 2 l/min measured by the flow rate sensor 100, and for an NO dosage of 5 ppmv, the mean flow rate of NO is 0.1 ml/min, and it increases when the minute ventilation increases and/or the NO dosage increases. The mean flow rate of NO can therefore vary from 0.1 to 45 ml/min.

In order to take into account this large flow rate amplitude, as has already been mentioned, the emergency dosing system 200 is provided with a first and a second calibrated orifice device 208, 209, which are arranged on the first and second dosing lines 206, 207 arranged downstream of the pneumatic regulator 204.

As is illustrated in FIG. 4, the first calibrated orifice device 208 is configured to generate relatively low flow rates over a wide pressure range, for example flow rates of below 10 ml/min., whereas the second calibrated orifice device 209 is configured to generate higher flow rates, which may exceed 50 ml/min, as is illustrated in FIG. 5.

However, over the 0-10 ml/min range, it can be seen that the relationship between the flow rate and the position of the stepping motor is unfavourable to the second calibrated orifice device 209 because a small variation in the position of the stepping motor 203 generates a large variation in flow rate, which can adversely affect the accuracy of the generated flow rates.

Consequently, the system is configured so that the gas flow passes through the first calibrated orifice device 208 when the mean flow rate of NO is low, that is to say less than or equal to 10 ml/min, and through the second calibrated orifice device 209 for higher mean flow rates of NO, i.e. above 10 ml/min.

The operating unit 130 therefore effects a specific control of the 3-way solenoid valve 205 according to the mean flow rate of NO in order to obtain a fluidic communication between its upstream port 201a and its first downstream port 205a (if flow rate <10 ml/min) or, if applicable, its second downstream port 205b (if flow rate >10 ml/min), in order to direct the emergency NO flow rate to the first dosing line 206 through the first calibrated orifice device 208 or, as the case may be, to the second dosing line 207 through the second calibrated orifice device 209.

During normal functioning, the operating unit 130 averages the NO flow rate and then uses the value thus determined to control the 3-way solenoid valve 205 in order to select the first or second dosing line 206, 207, therefore the first or second calibrated orifice device 208, 209, intended to receive the flow rate of NO, i.e. the one which will be used to operate the emergency dosing of NO, in the event of malfunction of the NO apparatus 1, in particular in the event of a fault in the electrical supply to the flow rate sensor 100 or to the operating unit 130.

Furthermore, the operating unit 130 performs a conversion by way of a stored lookup table or the like, taking into account the selected calibrated orifice, i.e. first or second calibrated orifice device 208, 209, so as to control the actuator 203 of the flow rate control device 210, such as a stepping motor, and to define a level of tension of the internal spring of the pneumatic regulator 204 in order to allow a flow rate of NO circulating in the emergency line 201 of the emergency system 200 that is equal to the calculated value of the fixed mean NO. This calculated value of mean NO therefore serves as emergency gaseous NO flow rate in the event of a malfunction of the apparatus 1.

During normal functioning, no gas flow circulates in the emergency line 201, because the all-or-nothing solenoid valve 202 is closed. The gas flow normally circulates in the injection line 111, via the proportional solenoid valve 113 and the flow rate measurement device 112, before being supplied to the NO injection module 110, which mixes the NO flow with the air flow or the like from the ventilator 2.

Therefore, in the event of a major failure of the NO delivery device 1 and/or of an interruption in its electrical supply, with the exception of the actuator 203, of the solenoid valve 205 and of the pressure regulator 115, which has a purely pneumatic function, all of the electromechanical actuators, in particular the solenoid valves, return to their rest position, due to the fact that the operating unit 130 is also not powered. In addition, the various sensors are without electrical supply and are therefore unable to communicate and/or to operate/control other components.

Thus, the proportional solenoid valve 113 recovers its rest position, namely its closed position preventing any passage of gas, while the solenoid valve 202 at the same time recovers its rest position, namely its open position, enabling passage of the gas coming from the NO source into the emergency line 201 of the emergency circuit 200, and its circulation as far as the second connection site 111b, then the downstream part of the injection line 111.

The NO/N₂ mixture then circulates in the emergency line 201 at the pre-fixed emergency flow rate which is controlled by the combination of the pneumatic regulator 204, in particular by its generated pressure, and the calibrated orifice selected, that is to say the first calibrated orifice device 208 or the second calibrated orifice device 209, knowing that, as has already been explained, the last valid value of the fixed mean flow rate of NO was determined by the operating unit 130 during the normal functioning of the device 1 prior to its malfunction.

The emergency flow rate of NO/N₂ which joins the injection line 111 (at 111b) can then be injected into the inhalation branch 31 of the patient circuit 3 via the NO injection module 110, as has already been mentioned.

In general, according to the invention, the flow rate control device 210 is configured to supply the gas, i.e. NO/N₂, at a pre-fixed emergency flow rate of gas, where said emergency flow rate of gas is determined by the operating means 130 one the basis of one or more gas flow rate measurements supplied by the flow rate measurement device 112 during a normal functioning of the device 1 prior to the malfunction, for example the last flow rate value that was measured before the malfunction that affects the correct functioning of the device 1.

The flow rate value is pre-regulated within the flow rate control device 210, for example by acting on the tension of the internal spring of the pneumatic regulator 204 of the flow rate control device 210, as has been explained above, by control, that is to say pre-regulation, of the flow control device 210 by the operating means 130. The pre-regulation takes place, that is to say is performed or carried out, during the normal functioning of the device 1.

Of course, using the emergency circuit 200 in the event of malfunction of the NO delivery apparatus 1 does not guarantee the same accuracy of the inhaled NO concentration as when the NO delivery system 1 is operating normally, that is to say by adjusting the flow rate of NO according to the flow rate passing through the flow rate sensor 100, but this avoids a break in supply of NO to the patient and, furthermore, the buffer volume generated by the portion of the inhalation branch 31 situated downstream of the NO injection module, which is optionally augmented by the volume of the humidification chamber when it is present, makes it possible to smooth the variations in the concentration of NO inhaled by the patient and to come close to the desired target value, that is to say the NO dosage.

The emergency dosing system of the invention is therefore particularly advantageous to implement because it increases the safety of the patient, who does not risk being deprived of treatment with NO, in the event of malfunction of the NO delivery apparatus, and who receives a dose of NO very close to, or even equal to, the desired dosage.

In other words, being able to come close to the desired target value of NO, by virtue of the emergency NO dosing system 200 integrated into the NO delivery device 1 of the invention, considerably improves the safety of the patient by comparison with a fixed emergency NO flow rate usually delivered by the safety system of the NO delivery devices of the prior art.

Thus, by way of comparison, with an emergency system based on a fixed flow rate, as conventionally implemented in the NO delivery devices of the prior art:
for a required mean NO flow rate of 0.05 l/min to normally ensure an NO concentration of 10 ppmv (case of use in neonatology with HFO ventilator), the resulting concentration with the fixed flow rate is 50 ppmv, which corresponds to a five-fold multiplication of the desired dosage.
conversely, for a required mean NO flow rate of 1 l/min to ensure 80 ppmv of NO concentration (case of use in adults, for example in case of pulmonary hypertension during cardiac surgery), the resulting concentration drops to 20 ppmv, which corresponds to a 75% reduction in the desired dosage.

In both cases, the considerable departures from the dosage can bring about situations that are unacceptable and dangerous for the patient, in contrast to the emergency NO dosing system 200 integrated into the NO delivery device 1 of the invention, which makes it possible to comply with the desired dosage.

It follows that the emergency NO dosing system 200 of the invention has undeniable advantages in enhancing patient safety by:
automatically injecting an emergency flow rate of NO without waiting for the user to realize the situation and intervene by switching to the emergency pneumatic dosing.
ensuring that the concentration of NO inhaled by the patient is similar to the concentration desired by the physician, that is to say the desired dosage.

Of course, the switch-over to the NO emergency dosing system 200 of the invention is only temporary, that is to say it lasts only for the time needed to replace the faulty equipment or component that triggered the acoustic and/or visual alarm alerting the medical personnel.

In order to avoid ill-advised activation of the NO emergency dosing system 200, the operating unit 130 is additionally configured to carry out suitable initialization and switch-off sequences. For example, if the NO therapy is deliberately stopped by the user, the operating unit 130 can control the actuator 203 in order to close the pressure regulator 204. Thus, in the event of deliberate switch-off and thus opening of the solenoid valve 202, the “closed” configuration of the pressure regulator 204 then prohibits any circulation of NO in the emergency line 201, for the period of time that the NO delivery device 1 is shut down.

The NO delivery device 1 equipped with the NO emergency dosing system 200 according to the invention is particularly suitable for supplying a gaseous mixture comprising 1 to 80 ppmv of NO and at least 20% by volume of oxygen, preferably at least 21% by volume of oxygen, to patients (adults, children, adolescents or neonates) suffering from pulmonary hypertension and/or hypoxia, which can cause pulmonary vasoconstriction or similar, for example caused by pulmonary diseases or disorders such as PPHN (persistent pulmonary hypertension of the newborn) or ARDS (acute respiratory distress syndrome), or those caused by heart surgery with placement of the patient on extracorporeal blood circulation.

What is claimed is:

1. NO delivery apparatus (1) for supplying an NO-containing gas, in particular an NO/N₂ gas mixture, comprising:
an NO injection line (111) for conveying the NO-containing gas,
a valve device (113) arranged on the injection line (111) in order to control the circulation of the NO-containing gas in the injection line (111), said valve device (113) being configured to be normally in a closed position for preventing any circulation of gas in the injection line (111),
a flow rate measurement device (112) arranged on the injection line (111) in order to perform one or more measurements of the flow rate of the NO-containing gas circulating in the injection line (111),
an emergency circuit (201) comprising an emergency line (201) which connects fluidically to the injection line (111), upstream (111a) and downstream (111b) of the valve device (113), said emergency line (201) comprising an emergency solenoid valve (202) configured to be normally in an open position in order to permit a circulation of gas in the emergency line (201), and a flow rate control device (210),
operating means (130) configured to cooperate with the emergency solenoid valve (202), the flow rate control device (210), the valve device (113) and the flow rate measurement device (112),
and in which, in the event of a malfunction causing a shut-down of cooperation with the operating means (130):
the emergency solenoid valve (202) is configured to pass to an open position in order to permit a circulation of gas in the emergency line (201) of the emergency circuit (200),
the valve device (113) is configured to pass to a closed position in order to stop any circulation of gas in the injection line (111), and
the flow rate control device (210) is configured to supply the gas at a pre-fixed emergency flow rate of gas, where said emergency flow rate of gas:
is determined by the operating means (130) on the basis of at least one gas flow rate measurement supplied by the flow rate measurement device (112), during a normal functioning of the apparatus (1) prior to said malfunction, and
is pre-regulated through command of said flow rate control device (210) by the operating means (130), during said normal functioning of the apparatus (1),
characterized in that:
a multi-way solenoid valve (205) is arranged on the emergency line (201), downstream of the flow rate control device (210),
said multi-way solenoid valve (205) comprising:
an inlet path fluidically connected to the emergency line (201) downstream of the flow rate control device (210),
a first outlet path fluidically connected to a first dosing line (206) comprising a first calibrated orifice device (208), and
a second outlet path fluidically connected to a second dosing line (207) comprising a second calibrated orifice device (209),
the first dosing line (206) and the second dosing line (207) are connected (201b) to the emergency line (201), downstream of said first and second calibrated orifice devices (208, 209),
and the operating means (130) are configured to operate the multi-way solenoid valve (205) to direct the gas flow towards the first dosing line (206) or, alternatively, towards the second dosing line (207).

2. Apparatus according to claim 1, characterized in that the multi-way solenoid valve (205) comprises 3 paths.

3. Apparatus according to claim 1, characterized in that the first calibrated orifice of the first calibrated orifice device (208) has a first passage diameter (D1) and the second calibrated orifice device (209) has a second passage diameter (D2) such that 1.5.D1<D2<4.D1.

4. Apparatus according to claim 3, characterized in that the first passage diameter (D1) and the second passage diameter (D2) are such that: 1.8.D1<D2<3.D1, preferably D2 is equal to about 2.D1.

5. Apparatus according to claim 1, characterized in that the emergency solenoid valve (202) is of the all-or-nothing type, able to assume only an open state, in which it allows the gas flow to pass, and a closed state, in which it interrupts the passage of the gas flow.

6. Apparatus according to claim 1, characterized in that the flow rate control device (210) comprises actuator means (203) cooperating with a pneumatic pressure regulator (204), and the actuator means (203) preferably comprises a stepping motor.

7. Apparatus according to claim 6, characterized in that:
the pneumatic regulator (204) is configured to be adjustable to several pressure levels between 0 and 2 bar relative, preferably less than 1.5 bar relative, and
the actuator means (203) cooperates with the pneumatic regulator (204) to set a desired output pressure downstream of said pneumatic regulator (204).

8. Apparatus according to claim 7, characterized in that:
the pneumatic regulator (204) comprises an internal spring for adjusting the desired pressure level, and
the actuator means (203) comprises a stepping motor configured to adopt several different angular positions, each angular position of the stepping motor corresponding to a given tension of the internal spring of the pneumatic regulator (204),
such that the desired output pressure level downstream of the pneumatic regulator (204) is determined by the tension of the internal spring of the pneumatic regulator (204) corresponding to the angular position adopted by the stepping motor.

9. Apparatus according to claim 1, characterized in that, during normal function, the operating means (130) are configured to operate the multi-way solenoid valve (205) to effect a fluidic communication between the inlet path of the multi-way solenoid valve (205) and either one of the first and second outlet paths of the multi-way solenoid valve (205) so as to have the gas flow pass through the first or second calibrated orifice device (208, 209).

10. Apparatus according to claim 1, characterized in that the operating means (130) comprise at least one microprocessor.

11. Apparatus according to claim 1, characterized in that the valve device (113) comprises a proportional solenoid valve.

12. Apparatus according to claim 1, characterized in that the flow rate measurement device (112) is configured to perform a plurality of successive flow rate measurements during normal functioning of the NO delivery apparatus (1).

13. Apparatus according to claim 1, characterized in that the operating means (130) are configured to determine the opening of the pneumatic pressure regulator (204) and/or the emergency flow rate on the basis of a lookup table stored by storage means, such as a computer memory.

14. Installation (1, 2) for supplying gas to a patient, comprising:
at least one NO source (250) containing an NO/N₂ gas mixture, preferably an NO/N₂ gas mixture containing between 100 and 2000 ppmv of NO, the remainder being nitrogen (N₂),
an NO delivery device (1) according to one of the preceding claims, supplied with NO/N₂ gas mixture by said at least one NO source (250),
an inhalation branch (31) of a patient circuit (3) supplied with NO/N₂ gas mixture by the NO delivery device (1), and
a medical ventilator (2) in fluidic communication with the inhalation branch (31) in order to supply said inhalation branch (31) with a respiratory gas containing at least 20% of oxygen, preferably air or an oxygen/nitrogen mixture.
